(19) United States
(12) Patent Application Publication
(10) Pub. No.: US 2016/0045113 A1
(43) Pub. Date: Feb. 18, 2016

(54) Ultraviolet Streptococcus Detection

(71) Applicant: Nicholas Michael Lorence, Bethesda, MD (US)

(72) Inventor: Nicholas Michael Lorence, Bethesda, MD (US)

(73) Assignee: Nicholas Michael Lorence, Bethesda, MD (US)

(21) Appl. No.: 14/460,693

(22) Filed: Aug. 15, 2014

(51) Int. Cl. A61B 5/00

(52) U.S. Cl. CPC A61B 5/0071; A61B 2503/06

(57) ABSTRACT

An ultraviolet light is used to rapidly differentiate bacterial versus viral causes of a sore throat by the amount of fluorescent light emitted, allowing for a rapid, painless, and simple means to diagnose strep throats. The invention is especially useful in diagnosing strep throat in children.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The present invention is in the technical field of detection of bacteria in human patients.

Sore throats are a common reason for physician visits, to diagnose as a viral or bacterial condition. Bacterial causes of sore throat are almost always caused by a streptococcus infection, particularly in children. If left untreated, strep throat can lead to numerous potentially serious complications, including rheumatic fever and inflamed kidneys. 9,000-11,500 cases of strep throat occur per year in the United States, and in current medical practice, cultures and rapid tests are used to determine whether or not the throat is infected bacterially by streptococci. Hence for distinguishing strep throat, there are key distinguishing features to differentiate between viral and bacterial causes of throat infections, for which this invention is ideally suited. To obtain a sample for both cultures and rapid tests, cotton swabs are used to swab the throat and are placed in both a rapid test and an overnight culture. Current rapid tests have 95 percent sensitivity, meaning that 5 percent of patients whose rapid tests return negative in fact do have a strep infection. This leads to a false assurance and can lead to others becoming infected due to the contagious nature of the bacteria. If patients do wait for a full culture return, it can take 1 to 2 days before it can be fully diagnosed and treatment can commence. Antibiotics are usually administered to treat strep throat, and are highly effective. Another limitation with current practice for both culture tests and rapid tests is the administration of the cotton swabs to the throat, especially with child patients. Many children feel a gagging sensation and have difficulty staying still during the administration of the swabs. Children ages 5-15 are most likely to have strep throat and their sensitivity with the administration of cotton swabs can lead to misdiagnosis. Difficulties with strep tests can lead doctors to obtain incomplete samples for testing.

Bacteria have a natural fluorescent ability. The aromatic protein amino acids in the cell walls of bacteria give off a rather strong ultraviolet (UV) fluorescence signal, which can be detected by shining UV light on the bacteria and seeing the resultant visible fluorescent light that is emitted.

### SUMMARY OF THE INVENTION

The present invention is a method of rapidly diagnosing streptococcal infections of the throat. It provides a solution for rapidly diagnosing strep throat without inducing a gagging feeling in the patient.

### DETAILED DESCRIPTION OF THE INVENTION

In an embodiment of the invention, a patient with a sore throat comes into the doctor's office, and a doctor or nurse shines an ultraviolet light into the patient's mouth directed at the patient's throat. Based off the degree of brightness in the back of the throat, the doctor or nurse can determine if the infection is viral due to a low brightness or bacterial if the throat is bright from fluorescence. A bacterial infection indicates strep throat and the patient then requires treatment with antibiotics. In a preferred embodiment of the invention, the patient described in the preceding paragraph is a child. In another embodiment of the invention, the patient's sore throat is painful and inflamed. With the usage of ultraviolet light, streptococcus bacteria can be detected in a patient's throat. This can lead to a quicker and easier diagnosis of strep throat, an improvement from the current use of bacterial cultures. This also allows the physician to determine effortlessly if the inflamed throat is due to a virus or bacteria. This leads to quick and accurate diagnosis, helping the patient. The invention is especially useful for diagnosing strep throats for children, because conventional strep tests cause many to gag and are often hard to administer to fidgety children.

One skilled in the art can determine which ultraviolet wavelength to shine into a patient's throat by taking plates of streptococcus bacteria and shining different wavelengths on the plates. The best wavelength to use when administering the test to patients will be the one that causes the streptococci to emit the most light/shine brightest.

While the foregoing written description of the invention enables one of ordinary skill to make and use what is considered presently to be the best mode thereof, those of ordinary skill will understand and appreciate the existence of variations, combinations, and equivalents of the specific embodiment, method and examples herein. The invention should therefore not be limited by the above described embodiment, method, and examples, but by all embodiments and methods within the scope and spirit of the invention as claimed.

### DEFINITIONS

The term “strep throat” refers to a bacterial throat infection caused by streptococcal bacteria commonly associated with a sore throat.

The following are claimed:

1. A method of diagnosing an infection of the throat by streptococcus bacteria in a human, comprising shining an ultraviolet light in the back of the throat of the human, and observing the degree of fluorescent brightness to determine whether the sore throat is caused by a virus or streptococcus bacteria.

2. A method as in claim 1, wherein the human has complaints of a painful sore throat.

3. A method as in claim 1, wherein the human is a child.
